(19) United States
(12) Patent Application Publication
(10) Pub. No.: US 2014/0045937 A1
(43) Pub. Date: Feb. 13, 2014

(54) METHODS OF TREATING POLYCYSTIC OVARIAN SYNDROME USING CHLOROGENIC ACID

(71) Applicant: Oculus Innovative Sciences, Inc., Petaluma, CA (US)

(72) Inventor: James J. Schutz, Petaluma, CA (US)

(21) Appl. No.: 13/960,999

(22) Filed: Aug. 7, 2013

Related U.S. Application Data
(60) Provisional application No. 61/680,769, filed on Aug. 8, 2012.

(51) Int. Cl. A23L 1/30; A61K 31/198; A61K 31/216

(52) U.S. Cl. CPC A23L 1/3002; A61K 31/216; A61K 31/198
USPC 514/533; 514/562

(57) ABSTRACT

The invention provides a method of treating or preventing polycystic ovarian syndrome (PCOS) in a mammal, comprising administering chlorogenic acid in an amount effective to treat or prevent PCOS in the mammal. In an embodiment of the invention, the method further comprises administering L-cysteine to the mammal.

### CROSS-REFERENCE TO RELATED APPLICATION

This patent application claims the benefit of U.S. Provisional Patent Application No. 61/680,769, filed Aug. 8, 2012, which is incorporated by reference.

### BACKGROUND OF THE INVENTION

Polycystic ovarian syndrome (PCOS) is a hormonal disorder among women of reproductive age. Patients with PCOS may experience any one or more of infrequent or prolonged menstrual periods, excess hair growth, acne, obesity, and ovarian cysts. Possible complications of PCOS may include any one or more of diabetes, high blood pressure, high cholesterol, endometrial cancer, infertility, and breast cancer.

In spite of considerable research into methods of treating PCOS, there still exists a need for improved methods for treating PCOS.

### BRIEF SUMMARY OF THE INVENTION

An embodiment of the invention provides a method of treating or preventing PCOS in a mammal, the method comprising administering chlorogenic acid in an amount effective to treat or prevent PCOS in the mammal.

### DETAILED DESCRIPTION OF THE INVENTION

It has been discovered that chlorogenic acid treats or prevents PCOS. Accordingly, an embodiment of the invention provides a method of treating or preventing polycystic ovarian syndrome (PCOS) in a mammal, the method comprising administering chlorogenic acid in an amount effective to treat or prevent PCOS in the mammal.

Chlorogenic acid (also known as 3-caffeoylquinic acid, chlorogenate, 3-O-caffeoylquinic acid, heriguard, and 3-(3,4-dihydroxycinnamoyl)quinic acid) is generally present in the leaves or fruits of dicotyledonous plants (for example, Rosaceae fruits such as apple, pear, peach, coffee bean, cacao bean, seed of grape, and artichoke). Chlorogenic acid may be chemically described as (1S,3R,4R,5R)-3-[(E)-3-(3,4-dihydroxyphenyl)prop-2-enoyl]oxy-1,4,5-trihydroxycyclohexane-1-carboxylic acid. Chlorogenic acid has a molecular formula of C₁₆H₁₈O₉, a molecular weight of 354.30872, and has the following chemical structure:

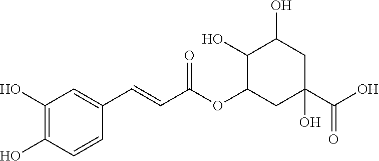 [SMILES: O=C(/C=C/c1ccc(O)c(O)c1)OC1CC(O)(C(=O)O)CC(O)C1O]

The method may comprise administering chlorogenic acid in an amount effective to treat or prevent PCOS in the mammal. An “effective amount” or “an amount effective to treat” refers to a dose of chlorogenic acid that is adequate to prevent or treat PCOS. Amounts effective for a therapeutic or prophylactic use will depend on, for example, the stage and severity of the PCOS, the age, weight, and general state of health of the patient, and the judgment of the prescribing physician. The size of the dose will also be determined by the method of administration, timing and frequency of administration, the existence, nature, and extent of any adverse side-effects that might accompany the administration of chlorogenic acid, and the desired physiological effect. It will be appreciated by one of skill in the art that PCOS could require prolonged treatment involving multiple administrations, perhaps using chlorogenic acid in each or various rounds of administration. By way of example and not intending to limit the invention, the dose of the chlorogenic acid can be from about 0.2 gram or less to about 1.5 grams or more once, twice, or three or more times per day, preferably from about 0.5 gram to about 1.25 grams once, twice, or three or more times per day, and more preferably about 1 gram once, twice, or three or more times per day. In an embodiment of the invention, where the mammal to be treated is an obese female human, any of the doses of chlorogenic acid described herein (for example, 1 gram of chlorogenic acid) may be administered multiple times per day (for example, two, three, four, or more times per day.

For purposes of the invention, the amount or dose of the chlorogenic acid administered should be sufficient to effect a therapeutic or prophylactic response in the mammal over a reasonable time frame. For example, the dose of the chlorogenic acid should be sufficient treat PCOS in a time period of from about 2 hours or longer, e.g., about 12 to about 24 or more hours, from the time of administration. In certain embodiments, the time period could be even longer. The dose will be determined by the condition of the mammal (e.g., human), as well as the body weight of the mammal (e.g., human) to be treated.

The chlorogenic acid may be any suitable chlorogenic acid, in any form, and may be obtained in any suitable manner. For example, the chlorogenic acid may be natural or synthetic.

In an embodiment of the invention, administering chlorogenic acid comprises administering a pharmaceutical composition comprising chlorogenic acid and a pharmaceutically acceptable carrier. The pharmaceutical composition comprising chlorogenic acid may be in any suitable dosage form and may be, for example, solid, semi-solid, gel, or liquid. Preferably, the pharmaceutical composition is in powder form.

In an embodiment, the method comprises adding chlorogenic acid to food and/or beverage for consumption. For example, the method may comprise administering a food and/or beverage comprising chlorogenic acid. In embodiments in which the chlorogenic acid is in powder form, the method may comprise mixing and/or dissolving the chlorogenic acid in a beverage and administering the beverage containing the mixed and/or dissolved chlorogenic acid to the mammal.

The pharmaceutically acceptable carrier may be any suitable pharmaceutically acceptable carrier. The carrier can be any of those conventionally used and is limited only by chemico-physical considerations, such as solubility and lack of reactivity with the active compound(s), and by the route of administration. The pharmaceutically acceptable carriers described herein, for example, vehicles, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use. The choice of carrier will be determined in part by the particular compounds used in the pharmaceutical composition, as well as by the particular method used to administer the chlorogenic acid.

In an embodiment of the invention, administering the chlorogenic acid to the mammal may comprise administering the chlorogenic acid orally, intravenously, intramuscularly, subcutaneously, or intraperitoneally. The following formulations for oral, intravenous, intramuscular, subcutaneous, or intraperitoneal administration are exemplary and are in no way limiting. More than one route can be used to administer the chlorogenic acid, and in certain instances, a particular route can provide a more immediate and more effective response than another route.

Oral formulations may include any suitable carrier. For example, formulations suitable for oral administration may comprise suitable carriers, such as lactose, sucrose, starch, talc magnesium stearate, crystalline cellulose, methyl cellulose, carboxymethyl cellulose, glycerin, sodium alginate or gum arabic among others.

In an embodiment of the invention, the carrier for the oral formulation may comprise one or more sweeteners. The sweetener may be any suitable sweetener as is known in the art and may be a natural or non-natural sweetener. Preferably, the sweetener is a natural sweetener. Exemplary sweeteners suitable for use in the present invention include any one or more of sucrose, stevia, and aspartame.

In an embodiment of the invention, the carrier for the oral formulation may comprise one or more flavorants. The flavorant may be any suitable flavorant as is known in the art and may be a natural or non-natural flavorant. Preferably, the flavorant is a natural flavorant. Exemplary flavorants suitable for use in the present invention include any one or more of lemon flavorant and cranberry flavorant. In an embodiment of the invention, the flavorant may comprise freeze-dried fruit, e.g., freeze-dried cranberry.

Formulations suitable for parenteral administration include aqueous and nonaqueous isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. The chlorogenic acid can be administered in a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations may comprise preservatives and buffers. In order to minimize or eliminate irritation at the site of injection, such compositions may contain one or more nonionic surfactants having, for example, a hydrophile-lipophile balance (HLB) of from about 12 to about 17. The quantity of surfactant in such formulations will typically range, for example, from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Injectable formulations are in accordance with an embodiment of the invention. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Toissel, 4th ed., pages 622-630 (1986)).

The pharmaceutical composition may comprise any suitable percentage of chlorogenic acid. In some embodiments, the chlorogenic acid is present in an amount ranging from about 5% or less to about 40% or more by weight of the pharmaceutical composition. In some embodiments, the chlorogenic acid is present in an amount ranging from about 10% to about 35% by weight of the pharmaceutical composition. In some embodiments, the chlorogenic acid is present in an amount ranging from about 15% to about 30% by weight of the pharmaceutical composition. In some embodiments, the chlorogenic acid is present in an amount ranging from about 20% to about 25% by weight of the pharmaceutical composition. Preferably, the chlorogenic acid it present in an amount of about 22% by weight of the pharmaceutical composition.

The pharmaceutical composition comprising chlorogenic acid may comprise any suitable amount of chlorogenic acid. In some embodiments, the pharmaceutical composition contains an amount of about 0.2 g or less to about 1.5 g or more of chlorogenic acid. In some embodiments, the pharmaceutical composition contains an amount of about 0.5 g of chlorogenic acid to about 1.25 g of chlorogenic acid. In some embodiments, the pharmaceutical composition contains an amount of about 0.2 g, about 0.5 g, about 1 g, about 1.25 g, or about 1.5 g of chlorogenic acid. Preferably, the pharmaceutical composition contains an amount of about 1 g of chlorogenic acid.

In an embodiment of the invention, the method further comprises administering L-cysteine to the mammal. L-cysteine is a non-essential amino acid and a precursor of the tripeptide glutathione (GSH). GSH is a peptide produced in the body that strengthens the immune system and which is composed of glycine, cysteine, and glutamic acid. L-cysteine has antioxidant properties which are typically expressed in GSH. L-Cysteine may be well absorbed in the body, may be converted to GSH quickly, and may be safer than other cysteine compounds, e.g., N-acetyl-L-cysteine (NAC).

It is believed that L-cysteine may enhance the PCOS therapeutic and/or prophylactic effect achieved by chlorogenic acid. NAC has been shown to treat or prevent PCOS in a variety of clinical trials as described in, e.g., Fulghesu et al., Fertil. Steril., 77(6): 1128-35 (2002); Salehpour et al., J. Obstet. Gynaecol. Res., e-published ahead of print Apr. 30, 2012; Oner et al., Eur. J. Obstet. Gynecol. Reprod. Biol., 159(1): 127-31 (2011); Saha et al., Fundam. Clin. Pharmacol., 26(1):54-62 (2012); Hashim et al., J. Womens Health, 19(11): 2043-8 (2010); Nasr, Reprod. Biomed. Online, 20(3): 403-9 (2009); Masha et al., J. Endocrinol. Invest., 32(11): 870-2 (2009); Rizk et al., Fertil. Steril., 83(2):367-70 (2005); and Fulghesu et al., Fertil. Steril., 77(6): 1128-35 (2002).

The L-cysteine may be any suitable L-cysteine, in any form, and may be obtained in any suitable manner. The L-cysteine may be natural or synthetic.

The method may comprise administering any suitable dose of L-cysteine. A suitable dose of L-cysteine will depend on, for example, the stage and severity of the PCOS, the age, weight, and general state of health of the patient, and the judgment of the prescribing physician. The size of the dose will also be determined by the method of administration, timing and frequency of administration, the existence, nature, and extent of any adverse side-effects that might accompany the administration of L-cysteine, and the desired physiological effect. By way of example and not intending to limit the invention, the dose of the L-cysteine can be from about 250 mg or less to about 1500 mg or more once, twice, or three or more times per day, preferably from about 500 mg to about 1200 mg once, twice, or three or more times per day, and more preferably about 600 mg once, twice, or three or more times per day. In an embodiment of the invention, where the mammal to be treated is an obese female human, any of the doses of L-cysteine described herein (for example, 600 mg of L-cysteine) may be administered multiple times per day (for example, two, three, four, or more times per day.

Although in some embodiments the method may comprise administering the chlorogenic acid and the L-cysteine sequentially, preferably the method comprises administering the chlorogenic acid and L-cysteine simultaneously. Although the simultaneous administration of chlorogenic acid and L-cysteine may comprise simultaneously administering a first pharmaceutical composition comprising chlorogenic acid and a second pharmaceutical composition comprising L-cysteine, preferably, the simultaneous administration comprises administering a single pharmaceutical composition comprising both chlorogenic acid and L-cysteine.

Accordingly, in an embodiment of the invention, the method comprises administering a pharmaceutical composition comprising chlorogenic acid, L-cysteine, and a pharmaceutically acceptable carrier. The pharmaceutical composition comprising chlorogenic acid and L-cysteine may be in any suitable dosage form and may be, for example, solid, semi-solid, gel, or liquid. The pharmaceutical composition may be as described herein with respect to other aspects of the invention.

In an embodiment, the method comprises adding chlorogenic acid and L-cysteine to food and/or beverage for consumption. For example, the method may comprise administering a food and/or beverage comprising chlorogenic acid and L-cysteine. In embodiments in which the chlorogenic acid and L-cysteine are in powder form, the method may comprise mixing and/or dissolving the chlorogenic acid and L-cysteine in a beverage and administering the beverage containing the mixed and/or dissolved chlorogenic acid and L-cysteine to the mammal.

The pharmaceutically acceptable carrier may be any suitable pharmaceutically acceptable carrier, and may be as described herein with respect to other aspects of the invention. The choice of carrier will be determined in part by the particular compounds used in the pharmaceutical composition, as well as by the particular method used to administer the chlorogenic acid and L-cysteine.

In an embodiment of the invention, administering the chlorogenic acid and L-cysteine to the mammal may comprise administering the chlorogenic acid and L-cysteine orally, intravenously, intramuscularly, subcutaneously, or intraperitoneally. Pharmaceutical formulations for oral, intravenous, intramuscular, subcutaneous, or intraperitoneal administration of chlorogenic acid and L-cysteine may be as described herein with respect to other aspects of the invention. More than one route can be used to administer the chlorogenic acid and L-cysteine, and in certain instances, a particular route can provide a more immediate and more effective response than another route.

The pharmaceutical composition comprising chlorogenic acid and L-cysteine may comprise any suitable percentage of L-cysteine. In some embodiments, the L-cysteine is present in an amount ranging from about 1% or less to about 30% or more by weight of the pharmaceutical composition. In some embodiments, the L-cysteine is present in an amount ranging from about 2% to about 25% by weight of the pharmaceutical composition. In some embodiments, the L-cysteine is present in an amount ranging from about 5% to about 20% by weight of the pharmaceutical composition. In some embodiments, the L-cysteine is present in an amount ranging from about 10% to about 15% by weight of the pharmaceutical composition. Preferably, the L-cysteine is present in an amount of about 11% by weight of the pharmaceutical composition.

The pharmaceutical composition comprising chlorogenic acid and L-cysteine may comprise any suitable percentage of chlorogenic acid. In some embodiments, the chlorogenic acid is present in an amount ranging from about 2% or less to about 40% or more by weight of the pharmaceutical composition. In some embodiments, the chlorogenic acid is present in an amount ranging from about 5% to about 35% by weight of the pharmaceutical composition. In some embodiments, the chlorogenic acid is present in an amount ranging from about 10% to about 30% by weight of the pharmaceutical composition. In some embodiments, the chlorogenic acid is present in an amount ranging from about 15% to about 25% by weight of the pharmaceutical composition. Preferably, the chlorogenic acid is present in an amount of 20% by weight of the pharmaceutical composition

The pharmaceutical composition comprising L-cysteine and chlorogenic acid may comprise any suitable amount of L-cysteine. In some embodiments, the pharmaceutical composition contains an amount of about 250 mg or less to about 1500 mg or more of L-cysteine. In some embodiments, the pharmaceutical composition contains an amount of about 500 mg of L-cysteine to about 1200 mg of L-cysteine. In some embodiments, the pharmaceutical composition contains an amount of about 250 mg, about 500 mg, about 600 mg, about 1200 mg, or about 1500 mg of L-cysteine. Preferably, the pharmaceutical composition contains an amount of about 600 mg of L-cysteine.

The pharmaceutical composition comprising L-cysteine and chlorogenic acid may comprise any suitable amount of chlorogenic acid. In some embodiments, the pharmaceutical composition contains an amount of about 0.2 g or less to about 1.5 g or more of chlorogenic acid. In some embodiments, the pharmaceutical composition contains an amount of about 0.5 g of chlorogenic acid to about 1.25 g of chlorogenic acid. In some embodiments, the pharmaceutical composition contains an amount of about 0.2 g, about 0.5 g, about 1 g, about 1.25 g, or about 1.5 g of chlorogenic acid. Preferably, the pharmaceutical composition contains an amount of about 1 g of chlorogenic acid.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount or any level of treatment or prevention of a PCOS in a mammal. Furthermore, the treatment or prevention provided by the inventive methods can include treatment or prevention of one or more conditions, complications, or symptoms of PCOS. Also, for purposes herein, “prevention” can encompass delaying the onset of PCOS, or a symptom, complication, or condition thereof.

The mammal referred to herein can be any mammal. As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

### EXAMPLE 1

This example demonstrates a pharmaceutical composition comprising chlorogenic acid and L-cysteine and a pharmaceutically acceptable carrier.

A powdered solid is prepared including the components set forth in Table 1.

TABLE 1

| Component | Amount |
|---|---|
| chlorogenic acid | 1 g |
| L-cysteine | 0.6 g |
| cranberry flavorant (freeze-dried cranberry) | 1 g |
| lemon flavorant | 1.6 g |
| stevia | 1.0 g |
| Total | 5.2 g |

The powdered solid is mixed in 8 ounces of water and administered to a human patient.

### EXAMPLE 2

This example demonstrates a pharmaceutical composition comprising chlorogenic acid and a pharmaceutically acceptable carrier.

A powdered solid is prepared including the components set forth in Table 2.

TABLE 2

| Component | Amount |
|---|---|
| chlorogenic acid | 1 g |
| cranberry flavorant (freeze-dried cranberry) | 1 g |
| lemon flavorant | 1.6 g |
| stevia | 1.0 g |
| Total | 4.6 g |

The powdered solid is mixed in 8 ounces of water and administered to a human patient.

### EXAMPLE 3

This example demonstrates method of treating PCOS and/or anovulation in a female patient, the method comprising administering chlorogenic acid and L-cysteine to the female patient. This example also demonstrates the effects of chlorogenic acid and L-cysteine on conception in women who are both anovulatory and afflicted with PCOS.

Ten female patients, between the ages of 25 and 37, each anovulatory, were evaluated. An anovulatory cycle is a menstrual cycle during which the ovaries do not release an oocyte. Chronic anovulation is a common cause of infertility. These patients were also attempting to become pregnant and presented with multiple symptoms of PCOS. Their diets were supplemented with GLUCOREIN medical food beverage (a powdered solid including the components set forth in Table 1 mixed in 8 ounces of water). Per instructions for use, the patients each consumed one eight-ounce serving of the GLUCOREIN beverage each day, typically in the morning hours.

After four months of treatment, with all ten patients continuing with the protocol, the results were as follows:

Three of the ten patients had conceived, all within two months of initiating treatment. None of these patients were treated with CLOMID (clomiphene). One patient miscarried. The other two pregnancies were progressing normally.

Of the seven patients who had not yet conceived after four months of treatment, two of those had demonstrated more regular menstral cycles and had tested positive for ovulation.

Of the five who had not begun to ovulate or demonstrated regular menses, three of them had body mass indexes of over 35. CLOMID (clomiphene) was added to their treatment regimen after three months of treatment, in an effort to incite ovulation, though none had yet tested positive for ovulation after four months of treatment.

One patient with a body mass index of 40 presented with painful menses. The patient underwent surgery four months after treatment began. The patient was diagnosed and treated for endometriosis as well as PCOS. Her ovaries presented as polycystic, though the GLUCOREIN beverage had been added to her diet four months earlier.

All ten patients continued with the daily protocol of GLUCOREIN beverage. No adverse side effects were noted.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating or preventing polycystic ovarian syndrome (PCOS) in a mammal, the method comprising administering chlorogenic acid in an amount effective to treat or prevent PCOS in the mammal.

2. The method of claim 1, wherein administering chlorogenic acid comprises administering a pharmaceutical composition comprising chlorogenic acid and a pharmaceutically acceptable carrier.

3. The method of claim 2, wherein the pharmaceutical composition is a solid pharmaceutical composition.

4. The method of claim 2, wherein the pharmaceutically acceptable carrier comprises one or more sweeteners.

5. The method of claim 2, wherein the pharmaceutically acceptable carrier comprises one or more flavorants.

6. The method of claim 1, comprising administering the chlorogenic acid orally.

7. The method of claim 1, comprising administering chlorogenic acid in a dose of about 0.2 g to about 1.5 g.

8. The method of claim 1, comprising administering chlorogenic acid in a dose of about 0.5 g to about 1.25 g.

9. The method of claim 1, comprising administering chlorogenic acid in a dose of about 1 g.

10. The method of claim 1, comprising administering a food and/or beverage comprising chlorogenic acid.

11. The method of claim 1, further comprising administering L-cysteine to the mammal.

12. The method of claim 11, comprising administering L-cysteine in a dose of about 250 mg to about 1500 mg.

13. The method of claim 11, comprising administering L-cysteine in a dose of about 500 mg to about 1200 mg.

14. The method of claim 11, comprising administering L-cysteine in a dose of about 600 mg.

15. The method of claim 11, wherein administering chlorogenic acid and L-cysteine comprises administering a pharmaceutical composition comprising chlorogenic acid, L-cysteine, and a pharmaceutically acceptable carrier.

16. The method of claim 15, comprising administering the pharmaceutical composition comprising chlorogenic acid, L-cysteine, and a pharmaceutically acceptable carrier orally.

17. The method of claim 15, wherein the pharmaceutical composition is a solid pharmaceutical composition.

18. The method of claim 17, wherein the pharmaceutical composition is in powder form.

19. The method of claim 15, wherein the pharmaceutically acceptable carrier comprises one or more sweeteners.

20. The method of claim 15, wherein the pharmaceutically acceptable carrier comprises one or more flavorants.

21. The method of claim 11, comprising administering a food and/or beverage comprising chlorogenic acid and L-cysteine.
